(19) United States
(12) Patent Application Publication
(10) Pub. No.: US 2020/0116163 A1
(43) Pub. Date: Apr. 16, 2020

(54) FAN MOUNTED ON A MONITOR OR TELEVISION

(71) Applicant: Darrin Niemiec, Schaumburg, IL (US)

(72) Inventor: Darrin Niemiec, Schaumburg, IL (US)

(21) Appl. No.: 16/599,998

(22) Filed: Oct. 11, 2019

Related U.S. Application Data
(60) Provisional application No. 62/745,123, filed on Oct. 12, 2018.

(51) Int. Cl. F04D 29/64; F04D 19/00; A61L 2/10

(52) U.S. Cl. CPC F04D 29/646; F05B 2240/91; A61L 2/10; F04D 19/002

(57) ABSTRACT

The present disclosure relates to a system for managing heat and temperature. In particular, the disclosure includes fan modules which may be mounted on a monitor or tv, wherein the fan modules include fans for moving air. The airflow from the fans may be used for the comfort of a user of the screen or monitor, or the airflow may optionally be used to dissipate heat generated from the screen or monitor. In additional embodiments, the fan modules may include a baffle, which defines an airway. A UV-C light source may be positioned within the airway, wherein the UV-C light source acts to decontaminate air as it moves through the airway.

### CLAIM OF PRIORITY

This application claims priority from Provisional Patent Application Ser. No. 62/745,123.

### CROSS REFERENCE TO RELATED DOCUMENTS

Priority is further herewith claimed under 35 U.S.C. § 120 from co-pending patent application Ser. No. 16/535,318 Filed an Aug. 8, 2019 which is a continuation of application Ser. No. 16/157,874 filed on Oct. 11, 2018 which issued as U.S. Pat. No. 10,316,141 which is a continuation-in-part of application Ser. No. 16/040,189, filed on Jul. 19, 2018, which is a continuation-in-part of application Ser. No. 15/589,367, filed on May 8, 2017, which is a continuation-in-part of application Ser. No. 15/471,762, filed an Mar. 28, 2017 issued as U.S. Pat. No. 10,006,616, which claims priority from Provisional Patent Application Ser. No. 62/439,719 filed Dec. 28, 2016. The disclosure of the co-pending patent application Ser. No. 16/535,318 is incorporated by reference herein in its entirety.

### FIELD OF THE INVENTION

The present invention relates to a fan system mounted on a monitor using a hinge. The fan operates to provide a pleasant work environment.

The present invention may incorporate an optional MERV or HEPA filter to remove odors, dust, debris, mold, allergens, germs and bacteria from the environment in proximity to the monitor, or, the fan may be configured to incorporate a UVC light source.

### SUMMARY OF THE INVENTION

The present invention relates to a fan system mounted on a monitor, display or television.

The present invention provides a fan device that is mounted to a monitor to create a pleasant environment in the vicinity of the monitor. The fan device not only moves the air to provide comfort, but the device can be outfitted with a heater to increase the comfort level for persons using the monitor.

The present invention may include the benefits of adapting the fan with an LED lighting fixture to fit into the footprint of a side panel of a monitor to permit installation of the monitor or a television.

The inventions further provides an air purifying device, comprising: a mount or side panel having at least one vent; a fan mounted to the mount or side panel; a baffle defining at least a first airway between the fen and the vent; and at least a first UV light source mounted in the first airway, wherein the first airway accommodates a UV-reflective material in at least a portion of the first airway; and wherein a first UV-screen is attached to the first airway to block UV light from exiting the airway.

The present invention also may provide an air purifying device comprises at least a second vent, and wherein the baffle further defines at least a second airway between the fan and the second vent, wherein a second UV light source is mounted in the second airway, wherein the second airway accommodates a UV-reflective material in at least a portion of the second airway, and wherein a second UV-screen is attached to the first airway to block UV light from exiting the airway.

Further yet, in some embodiments the UV-reflective material creates a kill zone which decontaminates air flowing through the first and second airway In some embodiments, a second fan mounted to the fan module and in-line with the first fan. Some embodiments include an air diversion mechanism configured to divert air into the first and second away. The first and second fan can be configured to rotate in opposite directions. The UV light source may be activated and de-activate remotely to decontaminate airflow through the first and second airway. In some embodiments, the UV light source is a UV-C light source having a wavelength between 200 to 280 nanometers.

The present invention further addresses the need to contain the light emitted from a UV-C light source within the chamber to create the kill zone. An extensive system of barriers is utilized within the kill chamber to create a kill zone while precluding the UV-C light from ex ting the kill chamber. The baffles may be coated with a reflective material to enhance the effectiveness of UV-C light within the kill chamber.

These and other objects and advantages of the present invention, as well as the details of the illustrative embodiments, will be more fully understood from the drawings and the description of the preferred embodiments.

### BRIEF DESCRIPTION OF THE DRAWINGS

The fan device of the present invention is described in more detail with reference to the attached drawings.

FIG. 1 is a front view of one of the embodiments of the fan module mounted on a monitor;

FIG. 2 is a top view of one of the embodiments of the fan module mounted on a monitor;

FIG. 3 is a perspective view of one of the embodiments of the fan module mounted on a monitor;

FIG. 4 is a perspective view of one of the embodiments of the fan module mounted on a monitor where the fan is rotated toward a rearward position;

FIG. 5 is a rear view of one of the embodiments of the fan module mounted on a monitor;

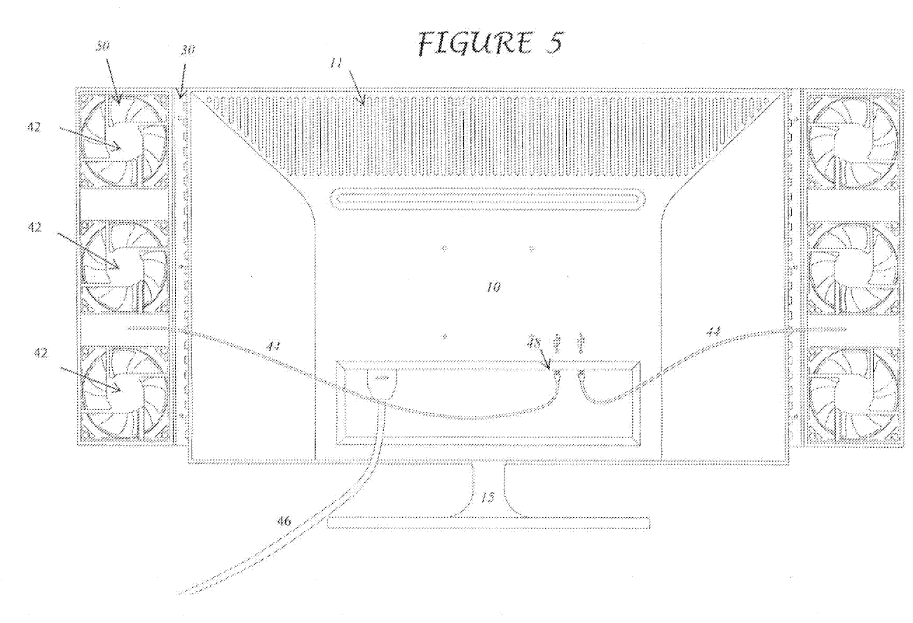

FIG. 6 is a front view of a second embodiment of the fan module mounted on a monitor wherein the fan module includes LED lights and a UVC kill chamber;

FIG. 7 is a top view of a second embodiment of a fan module shown in a cut-away, sectional view, attached to a monitor wherein the fan module includes LED lights and a UVC kill chamber; and

FIG. 8 is a top view of a fan module shown in a cut-away view depicting the UVC kill chamber.

### DETAILED DESCRIPTION OF THE PREFERRED EMBODIMENTS OF THE INVENTION

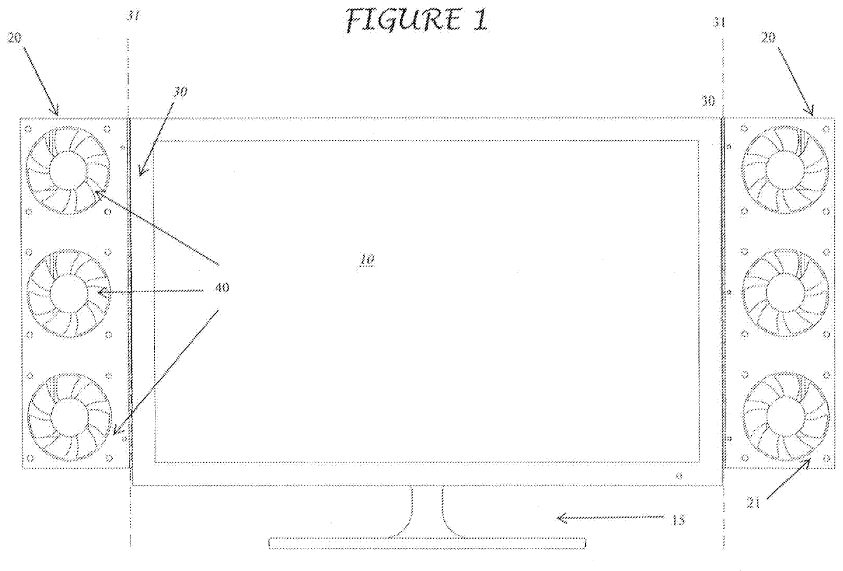

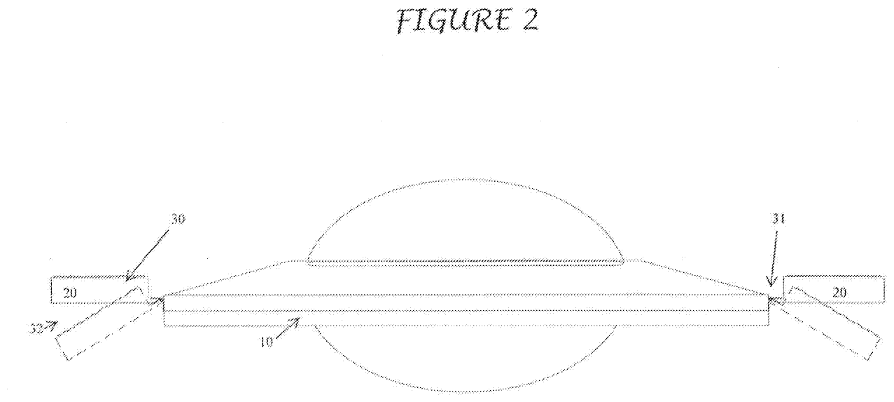

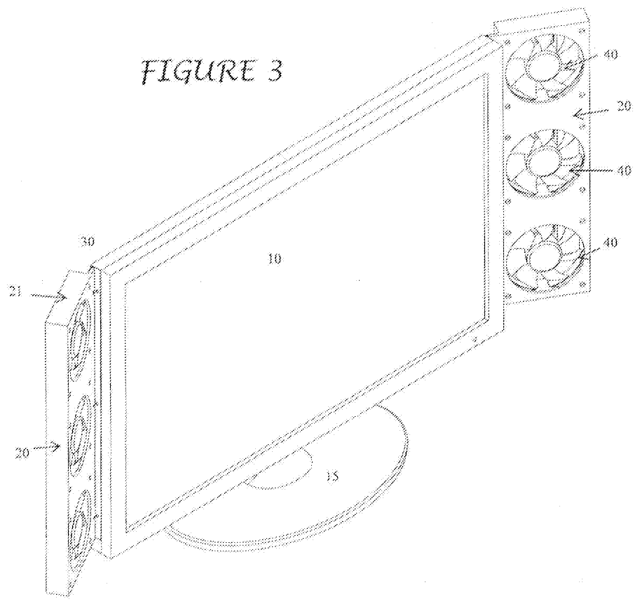

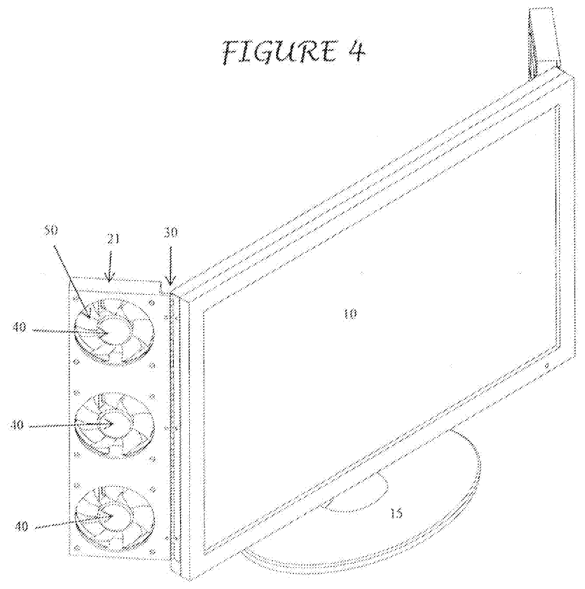

The fan device of the present invention is intended to be mounted on either a computer display, a monitor or television. The fan device is shown in the various drawings FIGS. 1-4 depict an embodiment of the present invention. The monitor 10 may be a computer screen, a television, a video screen or a monitor. Attached to the monitor 10 are a pair of fan modules 20. The fan module 20 is mounted to the monitor 10 by means of one or more hinge members 30. The hinge members 30 permit the fan module 20 to be rotated 32 about a hinge axis 31. The monitor 10 may have a base 15.

FIG. 1 of fan module 20 can be mounted to a monitor 10 using a piano-style hinge 30. While the piano-style hinge 30 is shown as the preferred mounting mechanism, it should be understood that other types of mounting systems may be utilized without departing from the spirit of the invention. It is preferred that the mounting system incorporates a hinge 30 such that the fan device can be rotated 32 about the hinge axis 31 to a position desired by the person utilizing the monitor.

As shown in FIGS. 1, 2 and 3, the fan module 20 includes a housing 21. The fan module 20 incorporate one or more fans 40 in a housing 21. The housing 21 is rotatably 32 affixed to a monitor 10. The fans 40 may include adjustable vents 50 to direct the flow of air from the fan. The fan module 20 may include a control switch (not shown) to operate the fans and control the speed of the fan. The fan may be adjustable by remote control, computer operation, motion or voice activation. The operation of the fans 40 may be connected to the operation of the monitor 10 by a power cable such that it turns on and off in correlation to the operation of the monitor 10. The preferred fans 40 operate at full speed at 70 cubic feet per minute and has a decibel level of 20 or less. A typical human cannot hear the fans operational at 25 decibels at a distance of two feet

There may be more than one fan 40 mounted in the housing 21. If more than one fan 40 is utilized in the housing 40, it may be desirable to have adjacent fans to operate in the opposite direction.

The fan module 20 may include a small heating unit (not shown) to operate in conjunction with the fan 40. When a small heating unit is used, the fan 40 can operate to distribute the warmed air toward the person using the monitor 10. During the summer months, the heating unit would not be activated. Thus, the fan 40 would operate to have a cooling effect on a user. Alternatively, instead of utilizing a heating unit 60 the fan 40 and housing 21 may be configured to take advantage of the heat generated by the monitor 10 and distribute the heat in the direction of a user. The purpose of the fan 40 is to provide a cooling effect in a warm environment and a heating effect in a cold environment to optimize the comfort level of the user. The fan module 20 may be rotated 32 about the axis 31 to direct air from the fan 40 toward user of the monitor 10. The fan module 20 may be configured such that the fans 40 are positioned in proximity to a fan inlet 42. The fan inlet 42 could be positioned near an outlet 11 of the monitor 10 from which the heat generated within the monitor 10 is dissipated from the monitor 10 and distributed toward a user The fan module 20 can be positioned to direct air from the fan 40 toward a user.

The fan 40 may be powered by electrical energy. The fan module 20 may incorporate a power cord 44 to supply energy to the fan module 20. The monitor typically has an AC power cord 46 The power cord 44 could be plugged directly into the power source, the monitor, or into a USB plug 48 in the monitor 10.

The fan module 20 may also incorporate a MERV or MEPA filter in conjunction with the fan. The filter could be positioned near the vent 50. A MERV filter refers to a minimum efficiency report valve to provide air-cleaning properties of a filter. MERV filters rated between 1 to 4 control contaminates like pollen, dust mites, dust, fibers, etc. MERV filters rated between 5 to 8 control contaminates such as mold, spores, hair dander, pudding mix etc. HEPA filter is a type of mechanical air filter that forces air through a fire mesh that traps harmful particles, such as, pollen, pet dander, dust mites and smoke. The MERV and HEPA filters improve the environment of the user.

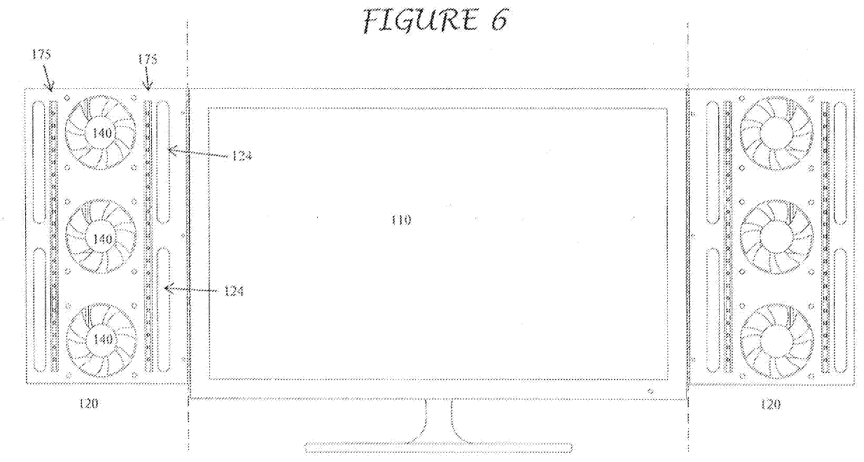

The fan module 20 may include an LED light source or a UV light source. U.S. Pat. No. 10,316,141 is directed to a built-in air flow mechanism and UV air purifying device built into a ceiling tile. This disclosure incorporates by reference the entirety of U.S. Pat. No. 10,316,141. Particular reference to specific examples, from U.S. Pat. No. 10,316,141, can be seen in FIGS. 6, 7 and 8 as well as Columns 13, Lines 17 through Column 16 line 47.

Embodiments of the invention further include the functionality of irradiating germs out of the air using UV light fixture positioned in the fan module. Such embodiments provide the advantage of not only circulating air in an environment, but also killing viral, bacterial, and fungal species which may be living in the environment's air. While the UV light degrades organic materials, inorganic materials (including metals or glass) are not affected by UV light. Therefore, UV light is effective for reducing organic matter which may be airborne in the air. Reducing airborne contaminants may be important in any environment, but especially in hospitals or schools, which may be particularly susceptible to disease. Regardless of the environment, disinfecting the air of contaminants is helpful to reduce the spread of disease.

It is preferable to reduce or eliminate contact with UV lighting because UV light can be harmful to humans and/or animals (particularly over prolonged durations). Embodiments of the invention therefore provide the advantage of positioning a UV light source in the fan module, where the UV rays may be contained in the fan module. For example. FIGS. 6 through 8 illustrate and depict a UVC light 170 source within the fan module 120 As shown in FIG. 6, the fan module 120 includes fans 140 which direct air into the fan module 120. The fan module 120 also include air vents 124 and LED light strips 175. UV light source(s) 170 mounted inside the upper baffle 122. The area 123 between the upper baffle 122 and the lower baffle 126 which is irradiated by the UVC light source 170 is referred to as the kilt chamber 123 and are pulled into the kill chamber 123 by action of the fan 140 The air is moved through a vent 124. A protective shield 125 may be positioned within the vent 124 or the kill chamber 123 to prevent light emitted from the UVC light source 170 from escaping from the kill chamber 123.

The UVC light source 170 emits light in a range that irradiates organic matter residing in air as air flows from the kill chamber 123 to the vent 124. A person of skill in the art would recognize that UV light sources include a power source and may optionally include an on/off controller (not shown). The UV light source 170 may be activated by an on/off button, or it may be controlled by the remote-control feature described further herein. In such an embodiment, a remote control may include the ability to activate or de-activate a UV tight source 170.

In some embodiments, light source(s) 170 may emit UVC light, which has a wavelength of approximately 200 to 280 nanometers. A person of skill in the art would recognize the UVC light is optimal for irradiating airborne contaminants (such as viruses, superbugs, mold, and the like) in most environments. In embodiments of the invention, the upper baffle 122 and/or the lower baffle 126 may be made of, or coated with, a UV-reflective material. A person of skill in the art would recognize that a UV-reflective material could include a metal, such as stainless steel, or a specialty coating. Lining the airway with a reflective material and/or reflective coating provides the advantage of creating a “kill chamber,” or “kill zone” inside the airways 123 where UV rays may bounce to increase their exposure to air passing through the kill chamber 123 and by extension, increase the irradiation of organic matter contained in the air.

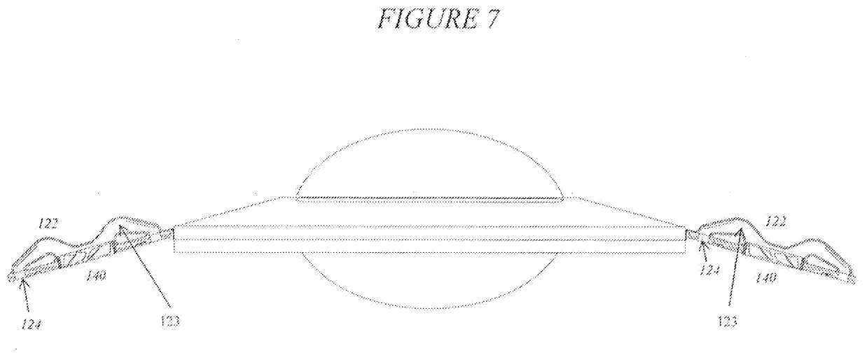

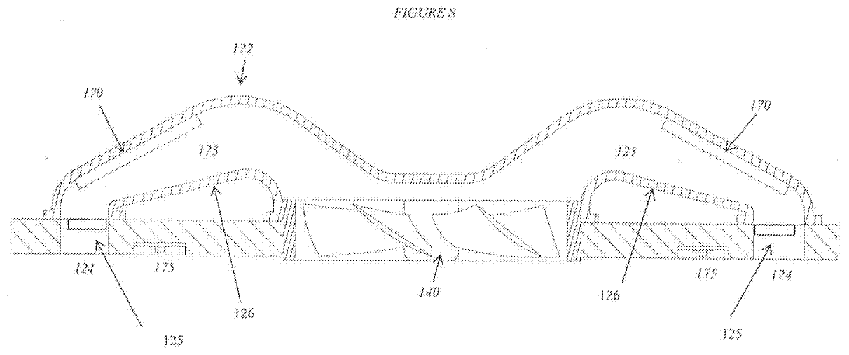

Furthermore, some embodiments of the inventions may include a UV-screen in the form of flange or protective shield 125 which may be positioned in the vent 124 or kill chamber 123 to shield UV rays from exiting the airways and entering an environment (such as a room or commercial space). In this way, including protective shield 125 in the vent 124 or kill chamber 123 prevents the UVC light rays from exiting the kill chamber 123. Although FIGS. 7 and 8 illustrate a UV source In an embodiment which is built into a fan module 120, it should be understood that the disclosed UV source and “kill chamber” may be implemented in any of the embodiments disclosed herein.

A test of a unit utilizing the UV-C light source 170 was conducted. The study was conducted to verify the unit's microbial reduction efficacy of aerosolized contaminants. The unit was mounted on the ceiling in a sealed 11′ 10″×11′ 10″×8′1″ (1125 cu.ft) controlled environment room. The unit's fan and UV lamps were powered on and allowed to warm up over the course of 2-hours as part of conditioning. Aliquots of the microorganisms were added to a pre-sterilized nebulizer reservoir. The testing room was sealed; all equipment activation was performed remotely. The nebulizer was powered to aerosolize the microbial suspension. Following 5 minutes, the UV-C right source and fans were powered on. Samples of the air were collected immediately after unit activation using Bio-aerosol air impinger (Biosampler, SKC, Inc.). The air sample were collected over the course of three minutes. Air samples were collected again following 1 and 2-hours following start. The system was deactivated, and the room was exhausted for 25 minutes before entry for sample retrieval and subsequent analysis. The study was repeated as described with only the fans running and then again with the unit completely powered off. All collected samples were analyzed in triplicate at the minimum as per standard lab operating procedures. Analysis was conducted as per laboratory—s accredited ISO17025:2005 methodology: bacteria were analyzed as per SM 9215 (APHA 2012) and MS-2 as per EPA 1602. Analysis was conducted using calibrated and/or validated Instruments to traceable standards (NIST). All QC was within method acceptance limit. No general environmental conditions are specified in the standard or have been identified that could affect the test results or measurements.

The test results demonstrated the following: The test resulted in a finding that 99.6% of K. pneumoniae was eliminated from the air after 1-hour of operation, and 99.998% of K. pneumoniae was eliminated from the air after 2-hours of operation. There was a 30% reduction of K. pneumoniae from the air after 1-hour of operation when the UV light source was not activated. The tests further found that 98.4% of the MSZ virus was eliminated from the air after 1-hour of operation and 99.6% of the MSZ virus was eliminated after 2-hours of operation There was a 27.2% reduction of MSZ virus from the air after 1-hour of operation when the UV light source was not activated.

While specific combinations of elements are disclosed in specific embodiments, it should be understood that any combination of the different features may be utilized in the combined fan.

The foregoing disclosure and description of the invention are illustrating and explanatory thereof, and various changes in the size, shape and materials as well as in the details of illustrated construction may be changed without departing from the spirit of the invention.

It is understood that the invention is not limited to the specific embodiments disclosed and that modifications and other embodiments are intended to be included within the scope of the appended claims. Although specific terms are employed herein, they are used in a generic and descriptive sense only and not for purposes of limitation.

1. A system for managing temperature from a monitor, comprising:
a monitor having a screen portion and a housing, the housing having a left hinge and a right hinge mounted to a left edge and a right edge of the housing, respectively; and
a first and second fan module respectively mounted to the left hinge and the right hinge;
wherein the first and second fan module each comprise a fan module housing, each fan module housing further comprising at least one fan mounted within a fan cut-out portion of the fan module housing.

2. The system of claim 1, wherein each fan module further comprises at least one vent positioned on a front surface of said fan module, and each fan module further comprises a baffle positioned on the back side of said fan module, the baffle defining at least one airway between the fan and the vent.

3. The system of claim 2, wherein the front surface of each fan module further comprises a second vent, and the baffle further defines a second airway between the second vent and the fan.

4. The system of claim 2, further comprising a UV-C light source mounted within each said airway.

5. The system of claim 4, wherein a portion of each said airway further comprises a kill chamber lined with a reflective material for reflecting UV-C light within the kill chamber.

6. The system of claim 4, wherein the UV-C light source emits light having a wavelength between 200 and 280 nanometers.

7. The system of claim 4, wherein the UV-C light source is configured to be activated remotely to decontaminate airflow through the airway.

8. The system of claim 1, further comprising:
at least one power cord connectable to a power outlet for powering the screen portion;
at least one USB outlet in the housing of the monitor; and
at least one USB cable for connecting the first and second fan module to the USB outlet to power the respective fan modules.

9. The system of claim 1, wherein the first and second fan module each comprise at least a first fan and a second fan, and wherein the first fan is configured to rotate in a first direction, and the second fan is configured to rotate in an opposite direction.

10. A method for managing temperature from a monitor, comprising:
providing a monitor having a screen portion and a house, the housing having a left hinge and a right hinge mounted to a left edge and a right edge of the housing, respectively; and
mounting a first and second fan module respectively to the left hinge and the right hinge;
wherein the first and second fan module each comprise a fan module housing, each fan module housing further comprising at least one fan positions on a front surface of the fan module housing.

11. The method of claim 10, further comprising the step of providing a baffle on the back side of said fan module, the baffle defining at least a first aim/ay between the fan and a vent on the front surface of said fan module.

12. The method of claim 11, further comprising the step of positioning a UV-C light source in said airway.

13. The method of claim 12, further comprising the step of lining the airway with a reflective material to create a kill chamber within the baffle, wherein UV-C light from the UV-C light source is reflected within the kill chamber.

14. The method of claim 12, further comprising the step of providing power to said fan and causing said fan to rotate in a first direction, said rotation of said fan causing air to flow through the airway between said vent and said fan.

15. The method of claim 12, further comprising the step of activating the UV-C light source to decontaminate the airflow through the airway.

16. The method of claim 12, wherein the UC-V light source emits light having a wavelength between 200 and 280 nanometers.

17. The method of claim 10, further comprising the step of rotating the first and second fan modules about the left hinge and the right hinge, respectively.

18. The method of claim 10, further comprising the step of plugging a USB cord into at least one USB outlet in the housing of the monitor, wherein the USB cord provides power to at least the first fan module.

19. The method of claim 12, further comprising the step of killing airborne pathogens in the airway by activating the UV-C light source.

20. The method of claim 10, further comprising the step of providing an LED strip light source on the front surface of said module.
